(19) United States
(12) Patent Application Publication
(10) Pub. No.: US 2023/0405370 A1
(43) Pub. Date: Dec. 21, 2023

(54) FULL HEAD ENCAPSULATION SOLAR POWERED RESPIRATOR

(71) Applicant: Nicholas J. Singer, Irvine, CA (US)

(72) Inventor: Nicholas J. Singer, Irvine, CA (US)

(21) Appl. No.: 18/456,885

(22) Filed: Aug. 28, 2023

Related U.S. Application Data
(63) Continuation-in-part of application No. 18/351,490, filed on Jul. 12, 2023.
Application No. 18/351,490 is a continuation of application No. 16/855,309, filed on Apr. 22, 2020, now Pat. No. 11,738,217.

(51) Int. Cl. A62B 18/04

(52) U.S. Cl. CPC A62B 18/04; A61M 16/1055; A61M 2205/7509

(57) ABSTRACT

A head encapsulation unit is disclosed which filters virus contaminated air. The head encapsulation unit may be deployed and cover the whole head of a user and have a transparent front layer with one or more filters attached to purify air to and from the interior volume of the unit. This head encapsulation unit allows for both medical personnel and infected individuals to better and more easily communicate than mouth covered filters, goggles and face shield while offering substantially more mitigation from spreading an infection or being infected. Additional secondary filters and one or more air pumps may be incorporated with the head encapsulation unit.

### CROSS-REFERENCE TO RELATED APPLICATIONS

This application is a continuation in part application of U.S. patent application Ser. No. 18/351,490, filed on 2023 Jul. 12, which is a continuation application of U.S. patent application Ser. No. 16/855,309, filed on 2020 Apr. 22, the entire contents of which are expressly incorporated herein by reference.

### STATEMENT RE: FEDERALLY SPONSORED RESEARCH/DEVELOPMENT

Not Applicable.

### BACKGROUND

The various aspects described herein relate to a head encapsulation unit for protecting a user from spreading or breathing in harmful viruses. The head encapsulation unit can be easily put on and sealed without any extensive training in the event of an emergency or pandemic.

Many devices exist in the marketplace that filter contaminated air so that the user does not inhale harmful viruses and bacteria that may be in the air. However, these devices suffer from certain deficiencies so that they still leave the user vulnerable to infection from the harmful virus. For example, these prior art devices may not form a sufficient seal with the person skin because of facial hair. The harmful virus may bypass the filtering mechanism of the device and be inhaled by the user through an air pathway formed at the interface between the device and the person's skin. Sometimes, a seal is broken between the device the person's skin when the user talks, smiles, coughs or sneezes. These facial movements break the seal and allow unfiltered air which may be contaminated to bypass the filtering mechanism of the prior art device. Moreover, these prior art devices leave the eyes and ears exposed to harmful viruses and bacteria (i.e., microorganisms). These and other prior art devices suffer from these and other deficiencies.

Accordingly, there is a need in the art for an improved device that a user can wear for filtering viruses and from the user spreading harmful viruses.

### BRIEF SUMMARY

The various aspects of the head encapsulation unit discussed herein relate to a device that can be worn over a person's head. The head encapsulation unit may be positioned on the user's head by way of a cap and a seal. The cap is disposed inside the head encapsulation unit and fits on top of the person's head. The seal is at a bottom portion of the head encapsulation unit and sealingly engages the person's neck. The cap and the seal positions the rest of the parts of the head encapsulation unit to the user's head and face. The head encapsulation unit has a body (i.e., housing, frame and front transparent layer) with cutouts. A filtering mechanism is mounted to the cutouts. The filtering mechanism allows the person to breathe filtered air since the filtering mechanism traps harmful contaminants such as viruses. The seal is easy to wrap around the user's neck and does not require extensive training and fitting to ensure that contaminated air is not transferred into the head encapsulation unit via the interface between the seal and the user's neck. Moreover, the frontal area of the head encapsulation unit may be transparent to allow the user to speak freely without fear of contaminated air seeping into the mask when the user speaks, makes a facial expression or through faulty fitting of the device to a user's anatomical features. The head encapsulation unit also when worn by an infected person would traps viruses in the head encapsulation unit so that the infected person is not spreading harmful viruses when the contagious.

More particularly, a head encapsulation unit for mitigating contact of an airborne virus from contacting mucous membranes of the eyes, nose and mouth is disclosed. The head encapsulation unit may comprise a body, a filter, and a seal.

The body may define an interior volume. The body may include a bottom portion, a transparent front panel having a first cutout, and a top portion.

The filter may be removably attachable to the first cutout.

The seal may be attached to the bottom portion of the body. The seal may include a strap, a base, and a cushion.

The strap may be removably attachable to the base. A through hole in the bottom portion of the body may be sufficiently large so that a person's head may be inserted through the through hole of the bottom portion so that the person can wear the head encapsulation unit. The cushion may provide a seal against a user's neck when the strap is pulled and attached to the base.

The body discussed herein may include a housing and a transparent layer. The body may be fabricated from a resilient and flexible material. The body may be resiliently biased to the expanded position and collapsible to a collapsed position.

The head encapsulation unit may further comprise a strap for holding the housing in the collapsed position for the purposes of storage and transportation. A first end of the strap may be attached to a first side of a frame of the body. A second end of the strap may be removably attached to a second side of the frame of the body.

The bottom portion of the body may be stretchable. In particular, the bottom portion of the body may be stretched out sufficient to allow the user to insert his or head into the inner volume of the body. The cushion of the seal may be fabricated from silicone, vinyl, neoprene or a closed cell foam. When the seal is closed (i.e., strap is tensions and attached to the base), then the cushion forms a seal to mitigate contaminated air from entering the inner volume of the body and the user from spreading viruses when contagious.

The housing may be resiliently biased to the expanded position. In this regard, the housing may be fabricated from a material which is resiliently biased to the expanded position. Alternatively or additionally, the housing may be formed with wire rods that are resiliently biased to form the housing in the expanded position. The wire rods can be bent so that the housing is collapsible to the collapsed position.

Additionally, a head encapsulation unit wearable by a user for mitigating contact to airborne viruses, bacteria, vapor, dust, and toxic emissions is disclosed. The head encapsulation unit may have a body defining an interior volume. The body may include a bottom portion, a transparent front panel having a first cutout and a second cutout, and a top portion. The head encapsulation unit may a first filter removably attachable to the first cutout and a second filter removably attachable to the second cutout. The head encapsulation unit may have an air pump attached to the first filter to provide ventilation to the interior volume of the body, the air pump powered by solar panels on the head encapsulation unit.

In some embodiments, the air pump of the head encapsulation unit may be a first air pump and a second air pump is attached to the second filter. In some embodiments, the first air pump may be an air blower pump and the second air pump may be an air vacuum pump. In some embodiments, the first air pump and the second air pump may be attached outside of the interior volume of the body. In some embodiments, the first air pump and the second air pump may be attached inside of the interior volume of the body. In some embodiments, the first air pump and the second air pump are configured to be remote controlled.

In some embodiments, the head encapsulation unit may have a third filter removably attachable to the first cutout and a fourth filter removably attachable to the second cutout. In some embodiments, the first and second filters may be designed to filter infectious airborne particles from entering the interior volume of the body and the third and fourth filters may be disinfectant filters. In some embodiments, the first and second filters may be closest to the interior volume of the body relative to the third and fourth filters. In some embodiments, the first and second filters are farthest away to the interior volume of the body relative to the third and fourth filters.

Furthermore, a head encapsulation unit wearable by a user for mitigating contact to airborne viruses, bacteria, vapor, dust, and toxic emissions is disclosed. The head encapsulation unit may have a body defining an interior volume. The body may include a bottom portion, a transparent front panel having a first cutout and a second cutout, and a top portion. The head encapsulation unit may have a first filter removably attachable to the first cutout and a second filter removably attachable to the second cutout. The head encapsulation unit may have a third filter removably attachable to the first cutout, and a fourth filter removably attachable to the second cutout. The first and second filters may be designed to filter infectious airborne particles from entering the interior volume of the body and the third and fourth filters may be disinfectant filters.

In some embodiments, the air pump of the head encapsulation unit may be a first air pump and the mounting bracket may be a first mounting bracket and a second air pump may be attached to a second mounting bracket that is over the second filter. In some embodiments, the first air pump may be an air blower pump and the second air pump may be an air vacuum pump. In some embodiments, the first air pump and the second air pump may be attached outside of the interior volume of the body. In some embodiments, the first air pump and the second air pump may be attached inside of the interior volume of the body. In some embodiments, the first air pump and the second air pump may be configured to be remote controlled.

In some embodiments, the first and second filters of the head encapsulation unit may be designed to filter infectious airborne particles from entering the interior volume of the body and the third and fourth filters may be disinfectant filters. In some embodiments, the first and second filters may be closest to the interior volume of the body relative to the third and fourth filters. In some embodiments, the first and second filters may be farthest away to the interior volume of the body relative to the third and fourth filters. In some embodiments the third and fourth filters may have potassium permanganate.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective view of a collapsed head encapsulation unit;

FIG. 2 is a perspective view of the head encapsulation unit shown in FIG. 1 as it is being traversed to an expanded position;

FIG. 3 is a perspective view of the head encapsulation unit shown in FIG. 1 when it is in the expanded position;

FIG. 4 illustrates filters and mounting brackets for attaching the filters to the head encapsulation unit;

FIG. 5 illustrates a release valve for protecting the user in the event that airflow through the filters is blocked;

FIG. 6 is a perspective view of the head encapsulation unit in the expanded position with the user inserting his or her head into the head encapsulation unit through a seal;

FIG. 7 is a perspective view of the head encapsulation unit being worn by a user;

FIG. 8 illustrates a strap removably attachable to a base for securing the seal against a user's neck;

FIG. 9 illustrates an elastic band that expandable and collapsible for allowing the user to put the head encapsulation unit on and seal a bottom portion of the unit to the user's neck;

FIG. 10 illustrates the incorporation of additional filters and one or more air pumps to the head encapsulation unit, where the one or more air pumps are on the outside of the unit; and

FIG. 11 illustrates the incorporation of additional filters and one or more air pumps to the head encapsulation unit, where the one or more air pumps are on the inside of the unit;

FIGS. 12A-B show the front and back view of an air blower pump;

FIGS. 13A-B show the front and back view of an air vacuum pump;

FIGS. 14A-B show respirator cartridges that may be used with the head encapsulation unit; and

FIG. 15 shows the incorporation of one or more solar panels with the head encapsulation unit.

### DETAILED DESCRIPTION

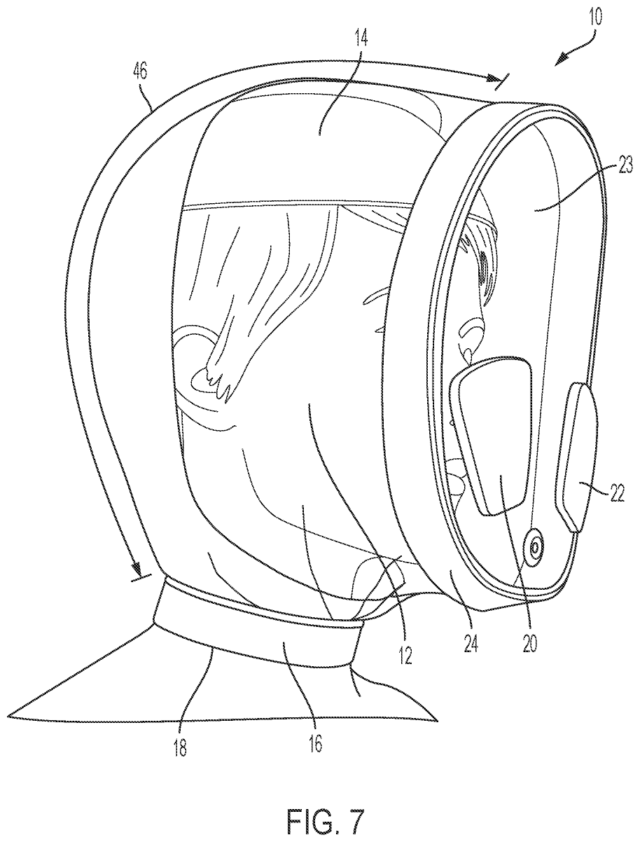

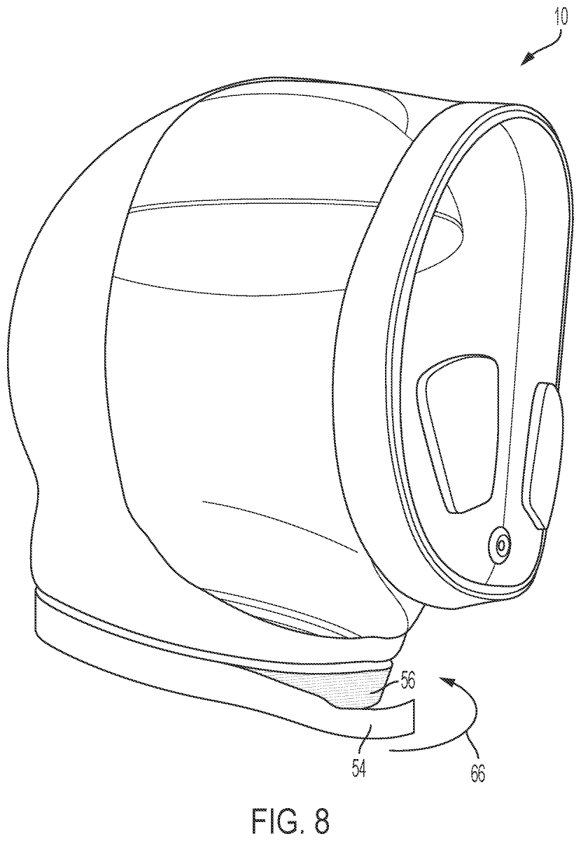

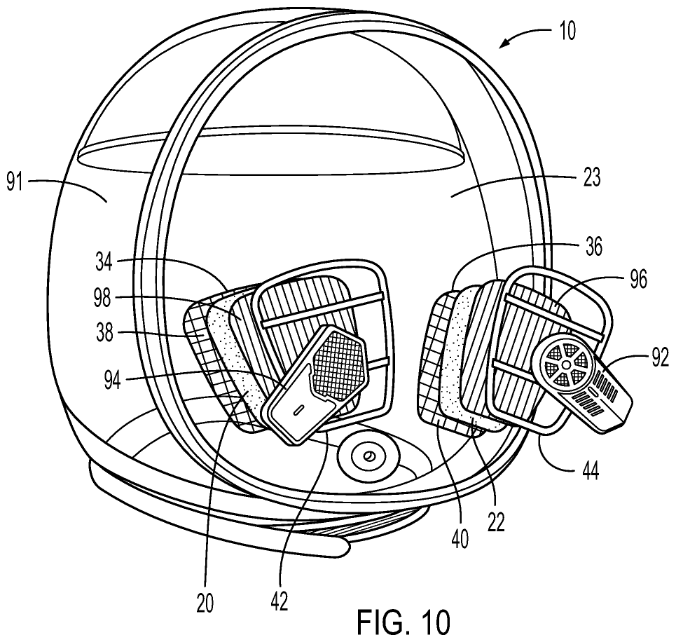

Referring now to the drawings, a head encapsulation unit 10 (FIG. 7) which can be worn on a person's head 12 is shown. The head encapsulation unit 10 may be positioned on the user's head 12 by way of a cap 14 and a removably attachable seal 16 that goes around the user's neck 18. The cap 14 and the seal 16 aligns the head encapsulation unit to the user's head. The head encapsulation unit 10 is also generally rigid when it is in the expanded position (FIG. 7) so that air can flow through filters 20, 22. The housing does not flex in and out as a user breathes in and out. Moreover, a front area and sides of the unit may be transparent so that the user can view his or her surroundings when wearing the head encapsulation unit. Because the head encapsulation unit covers the entire person's head, microorganisms, viruses, dust and other micro particulates do not enter the person's cavities (e.g., mouth, nasal, eyes, ear cavities). Rather, the user's entire head is protected from environmental organic and nonorganic materials. The filters 20, 22 may be selected to match the level of protected needed by the user. Moreover, when the user is infected with a harmful virus and contagious, the unit prevents the user from spreading the harmful virus. As shown in FIG. 10, additional secondary filters 96, 98 and one or more air pumps 92, 94 may be incorporated with the head encapsulation unit 10. The secondary filters 96, 98 provide an additional layer of filtration for the outside air entering the interior volume 91 of the head encapsulation unit 10. The one or more air pumps 92, 94 may be incorporated to improve the ventilation within the interior volume 91 of the head encapsulation unit 10.

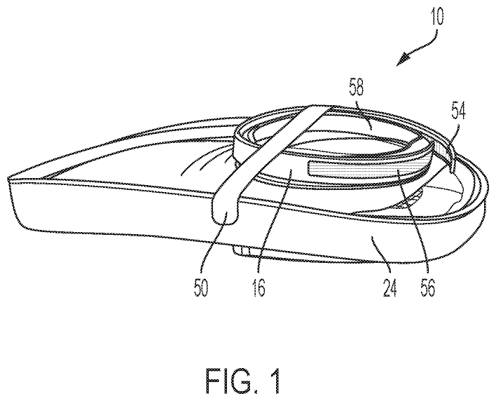

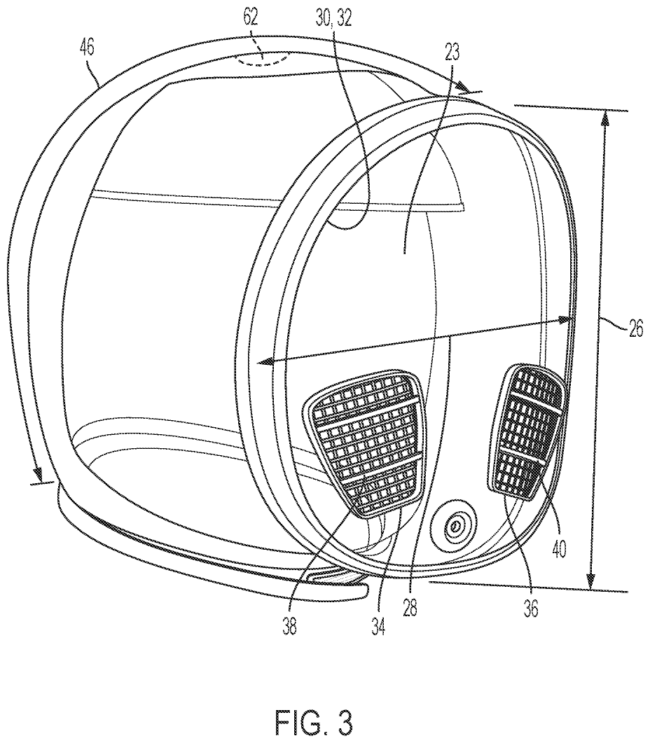

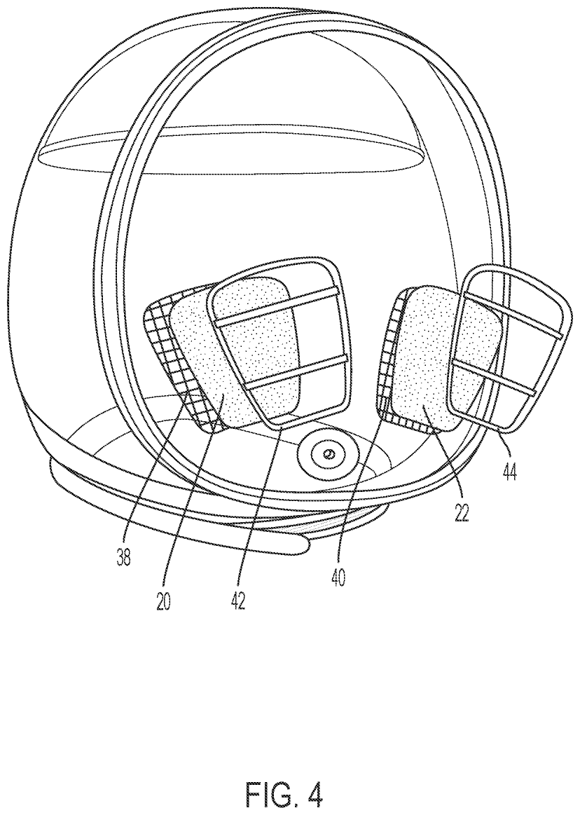

Referring now to FIG. 1, the head encapsulation unit 10 may be provided in a collapsed position. Moreover, the head encapsulation unit 10 may be traversed to the expanded position (FIG. 7). Moreover, the head encapsulation unit 10 may have a rigid frame 24 (FIG. 7). This rigid frame 24 may have a generally oval configuration with a height 26 (FIG. 3) between 8.3 inches and 14 inches but is preferably about 10.5 inches. The rigid frame 24 may also have a width 28 between 5.5 inches and 8 inches and is preferably about 7 inches. The frame 24 may be fabricated from a lightweight material including but not limited to plastic, polymer, composite, and other materials known in the art. Preferably, the rigid frame 24 has a porosity which is lower than the porosity of the filters 20, 22 and is preferably nonporous. The rigid frame 24 may hold its shape regardless of whether is traversed to the expanded or collapsed positions. The front of the rigid frame 24 may have a transparent layer 23. The transparent layer 23 may be fabricated from a nonporous material such as polyethylene, vinyl or plastic. The outer periphery 30 of the transparent layer 23 may be hermetically sealed to the inner periphery 32 of the rigid frame so that micro particulates do not pass from the environment to the interior volume of the head encapsulation unit 10 when worn.

The transparent layer 23 may also have two (2) cutouts 34, 36. These cutouts may receive mounting brackets 38, 40. These mounting brackets 38, 40 are also sealed to the inner periphery of the cutouts 34, 36 so that microorganisms (viruses and bacteria) and microparticulate do not pass from the environment into the interior volume of the head encapsulation unit 10. The filters 20, 22 may be removably mountable to the mounting brackets 38, 40. To aid in the removeable attachment of the filters 20, 22 to the mounting brackets 38, 40, mounting covers 42, 44 may cover the filters 20, 22 and be removably attachable to the mounting brackets 38, 40 via detents.

The filters 20, 22 which are mounted to the mounting brackets 38, 40 may be selected based on the particular use of the head encapsulation unit 10. For example, the filters 20, 22 mounted to the mountain brackets 38, 40 may be an N95 or an N99 filter. These filters are only exemplary and other filters having different ratings for differing sizes and concentration of micro particles may be utilized and mounted to the mountain brackets 38, 40.

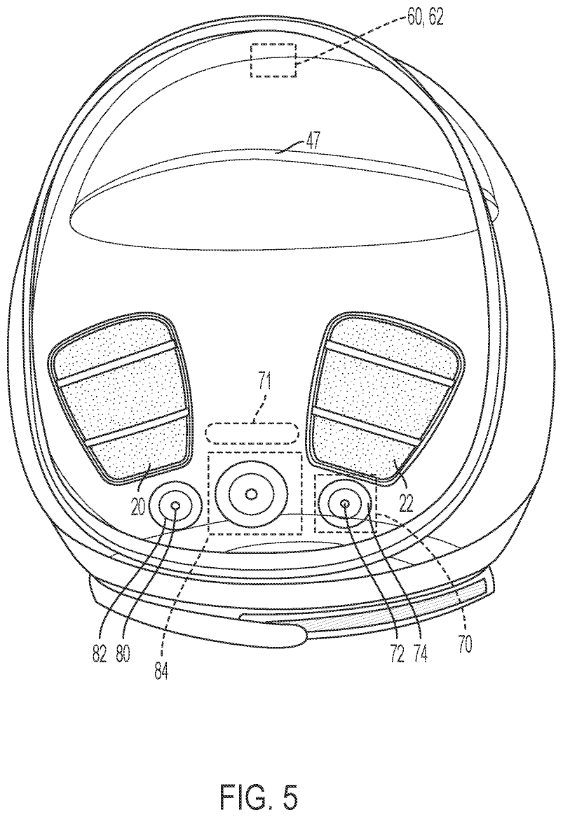

The filters 20, 22 may be disposed so that they're offset from the user's mouth. Referring to FIG. 5, the user's mouth 71 may be positioned in the middle of the two filters 20, 22. In this way, when the user's speaks, spit, sneezes, coughs, etc., microorganisms emanating from the user is less likely to contaminate the filters 20, 22. Plus, this area in front of the user's mouth 71 may be transparent to facilitate communication between the user and others.

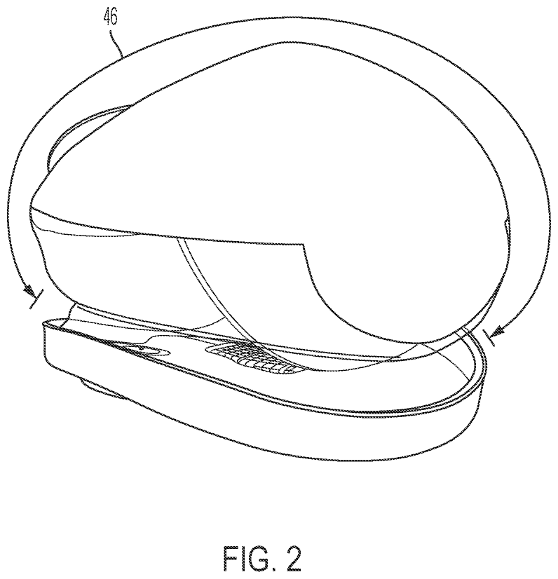

A housing 46 may be attached to the frame 24. The housing 46 may be fabricated from a material that is flexible so that the housing can be traversed to the collapsed position (FIG. 1) from the expanded position (FIG. 7). Moreover, the material from which the housing 46 is fabricated may also be biased in the outward position (FIG. 3). In this regard, in order to traverse the head encapsulation unit 10 from the collapsed position to the expanded position, the user can release an optional strap 50 (FIG. 1). When the strap 50 is released, the housing 46 opens up as shown in the transition illustrated in FIGS. 1-3. As described therein, the housing 48 may be fabricated from a flexible but resilient material that is biased to the expanded position to facilitate the transition of the housing 46 from the collapsed position to the expanded position. However, it is also contemplated that the housing may have thin wire rods that are shaped to the expanded position of the housing 46 and is bendable so that the housing is in the collapsed position. The material from which the housing 46 is fabricated may be a flexible material. The wire rods would be lined or embedded in the housing material so that the housing 46 can be transitioned from the collapsed to the expanded positions without any need to push open the housing with the user's hand. In this regard, when the strap 50 is released or open, the flexible but resilient wire rods do the work of traversing the housing 46 from the collapsed position to the expanded position.

To store the head encapsulation unit 10, the user may press the housing toward the frame 24 then close the strap 50. The strap 50 holds the housing 46 in the collapsed position for transport and/or storage of the head encapsulation unit 10. When the housing 46 is collapsed, the forces from the user's hand works against the outward biasing force of the material and/or the wire rods.

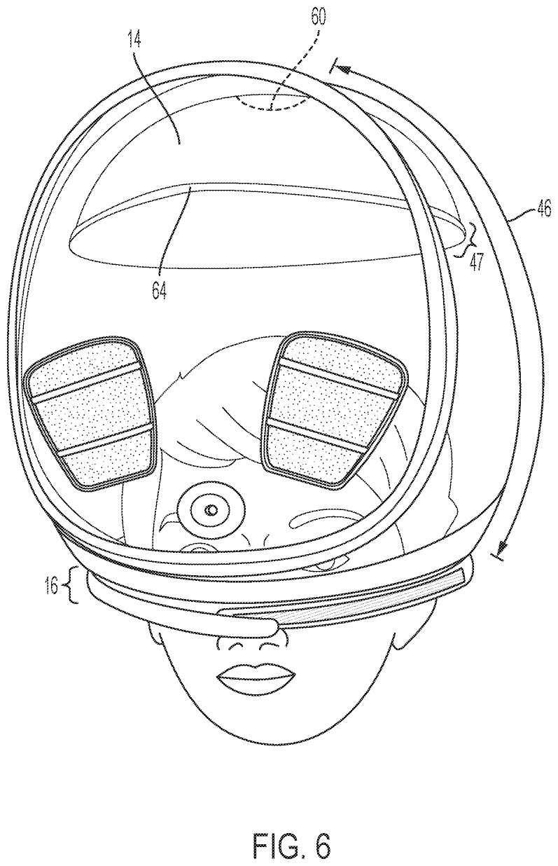

A bottom portion of the housing 46 may have a seal 16. This seal 16 may be expanded so that the user's head could pass through the seal and be disposed within the interior volume of the head encapsulation unit 10, as shown in FIG. 6. FIG. 6 illustrates the user's head at its widest position (i.e., at the ears) passing through the seal 16. Moreover, the seal 16 can be collapsed to a size of the user's neck 18, as shown in FIG. 7. The seal 16 may be hermetically sealed to the bottom portion of the housing 46. When the seal 16 is collapsed about the circumference of the person's neck, the seal 16 forms a seal with the person's skin to mitigate micro particulates from entering into the interior volume of the head encapsulation unit 10 as a person breathes in and from microorganisms getting out of the unit 10 in case the user is sick and contagious.

Referring now to FIG. 1, the seal may have a strap 54 and the base member 56 on the other end. The strap is removably attachable to the base member 56 by way of hooks and loops and other removably attachable mechanical devices. An inner side of the seal 16 may be lined with a nonporous cushion material 58 such as silicone, vinyl, neoprene or closed cell foam. When the strap 54 is attached to the base 56, the strap 54 places inward pressure so that the cushion 58 applies slight pressure to the person's neck and the cushion forms a seal with the person's neck.

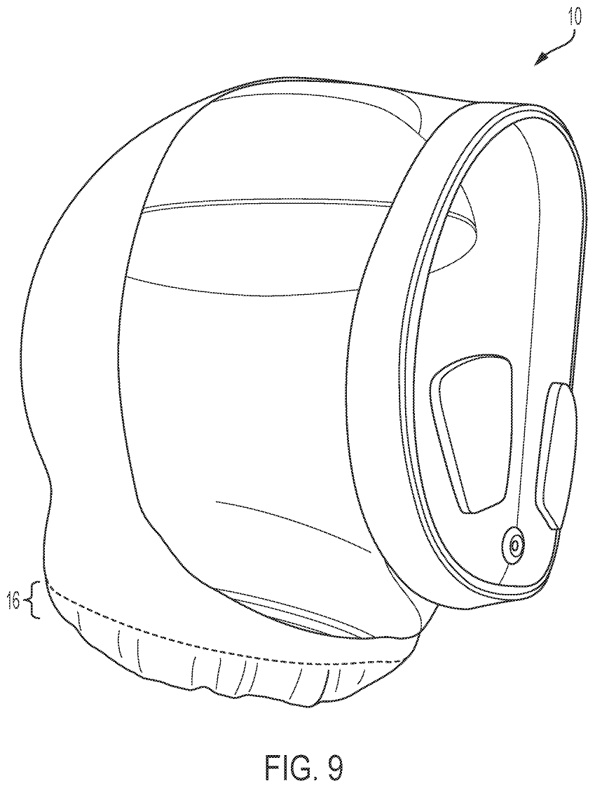

Alternatively, the seal may be an elastic band as shown in FIG. 9. The elastic band may be sewn into a bottom hem of the housing 46. The bottom portion of the housing when fully expanded can fit a person's head therethrough. Additionally, the elastic band can be stretched out to the fully expanded size of the bottom portion. The elastic band can also contract to the size of the person's neck (e.g., 13, 14, 15, 16 or 17 inches in diameter). The inner side of the seal may have the cushion as discussed herein. The pressure required to pass between the cushion and the person's neck may be greater than the pressure required to pass air through the filters 20, 22.

The housing 46 may have a cap 14. As discussed above, the cap 14 positions the head encapsulation unit on the person's head. The top portion 60 (FIG. 6) of the cap 14 may be secure to the top portion 62 (FIG. 3) of the housing 46 on its interior side, as shown in FIG. 5. The cap may be provided in various sizes to fit a person's head. By way of example and not limitation, the cap 14 may have a band 47 (FIG. 5) which defines a circumference 64 (FIG. 5). The band 47 may be provided in sizes having a circumference between 8 inches and 18 inches. The band 47 of the cap 14 may be elastic so that the circumference 64 may stretch between 8 to 9 inches, 9 to 10 inches, and so forth in 1 inch increments up to 18 inches. The head encapsulation unit 10 may be selected based on the user's head diameter about its forehead so that the cap of the head encapsulation unit fits the user's head.

Referring now to FIG. 6, as the user inserts his or her head through the seal 16 and the bottom portion of the housing, the user may insert his or her head into the cap 14. The cap may be fabricated from a material that is biased to the open position as shown in FIG. 6. When the housing 46 is traversed from the collapsed position to the expanded position, the cap 14 is also traversed from a collapsed position to the open position. When the user wears the cap 14 about his or her head, the seal is positioned at the user's neck. The strap 54 may be wrapped around the user's neck and attached to the base 56 as shown by arrow 66. To close the seal 16, the user may pull on the strap 54 slightly to generate a small force. The strap 54 may then be attached to the base 56. This action provides a slight pressure against the person's neck and the cushion of the strap. The cap 14 and the seal 16 positions the housing 56, the rigid frame 24 and the transparent layer 23 in the proper position so that the head encapsulation unit 10 is comfortable upon the user's head.

The head encapsulation unit 10 may also have a release valve 70. As shown in FIG. 5, the transparent layer 23 may be fitted with the release valve 70. The release valve 70 acts as a protective measure for the user. In the event that the filters 20, 22 are clogged and prevent air from entering or leaving the head encapsulation unit 10, the release valve 70 will burst to allow air to enter and leave the head encapsulation unit 10 to bring air into the head encapsulation unit 10 and to vent carbon dioxide out of the head encapsulation unit 10. The release valve 70 may be fabricated from a frangible material 72. The frangible material may have a burst pressure that is greater than the positive and negative pressures generated within the head encapsulation unit as the person's breaths in and out and the filters 20, 22 are operating within its normal operations. The frangible material may be centrally embedded within a button 74. The button 74 may be embedded into the transparent layer 23.

During use, the head encapsulation unit may be removed from a storage locker. To put the head encapsulation unit on, the user may release a first end of the strap from the frame. Because the housing 46 is biased to the expanded position, the housing begins to transition from the collapsed position (FIG. 1) to the expanded position (FIG. 3). Once the housing 46 is in the expanded position, the user may detach the strap of the seal from the base in the event that it is not already detached. With his or her hands, the user may stretch open the bottom portion of the housing so that the head encapsulation unit can be worn by the user. In particular, the user inserts his or her head into the through-hole formed at the bottom portion of the housing.

Additionally, once the head of the user is inserted into the head encapsulation unit, the user positions a cap of the head encapsulation unit onto the person's head. The cap was unfolded and traversed to the expanded position when the housing 46 was traversed to the expanded position. The cap positions the front transparent layer 23 at the proper distance in front of the user's face. The user can then tension the strap of the seal and attach the strap to the base of the seal. When the strap is attached to the base, a cushion on the interior side of the seal pushes against the skin at the neck of the user. Alternatively, if the unit 10 has the elastic band as shown in FIG. 9, the user may release the bottom portion to allow the elastic band to close the bottom portion, and thus the cushion on the user's neck. The cushion and the neck form a seal to mitigate contaminated air from entering into the inner volume of the head encapsulation unit during use. Also, the head encapsulation unit prevents the user from spreading viruses if and when the user is infected and contagious.

With the head encapsulation unit mounted to the user's head, the user may begin to breathe. When the user breathes in and out, the inner volume has a pressure that increases and decreases. Such pressure under normal circumstances is not sufficient to break the frangible material of the release valve. Plus, the pressure required to pass air through the cushion and the user's neck is greater than the pressure required to pass air through the filters. Additionally, the housing, the frame and the front transparent layer is sufficiently rigid so that the housing doesn't flex in and out but permits air to flow through the filter as the person breathes.

The head encapsulation unit allows the user to talk freely while maintaining a seal at the user's neck. The device does not require specialized fitting to ensure that the seal exists and is maintained during usage. Moreover, when the user speaks or makes facial expressions, since the seal interface is not between the user's face and the mask but the user's neck and the head encapsulation unit, the seal is not broken during normal talking or movements of the person's face.

Referring back to FIG. 5, the transparent layer 23 may have various features to assist the user in communicating with others, to treat the air within the head encapsulation unit and to allow the person to intake fluids. More particular, the area of the transparent layer directly in front of the user's mouth 71 may be fabricated with a thin flexible material. The thin flexible material is between about 1 inch by 1 inch to 4 inches by 4 inches. Since the material is thin and flexible, when the user speaks, the thin flexible material vibrates and allows the sound waves to pass through the thin flexible material. Additionally or alternatively, the transparent layer 23 may be fitted with a microphone 80 and a speaker 82. The microphone 80 may be positioned on an inner side of the transparent layer 23. The speaker 82 may be positioned on an outer side of the transparent layer 23. A battery may be connected to the microphone and speaker to drive the speaker and operate the microphone. Additionally, a speaker 86 may be disposed on the inner side of the transparent layer 23 and may be positioned adjacent to the user's ear. Plus, a receiver 88 (e.g., Bluetooth enabled) may be connected to the speaker. Other people may speak to the user by downloading an app to a smartphone. Other people can start the app then speak into the phone which transmits the voice data to the Bluetooth enabled receiver. The voice data is then communicated to the speaker 86 so that the user can listen to the other person.

A fluid input port 84 may be positioned centrally and immediately below the thin flexible area of the transparent layer 23. The fluid input port allows a user to insert a tube through the transparent layer. The tube may insert oxygen, atomized disinfectant and water. The tube may be operative to carry water so that the user can hydrate him or herself during use without having to remove the head encapsulation unit. The fluid input port may be valve which opens when the tube is pushed against the valve.

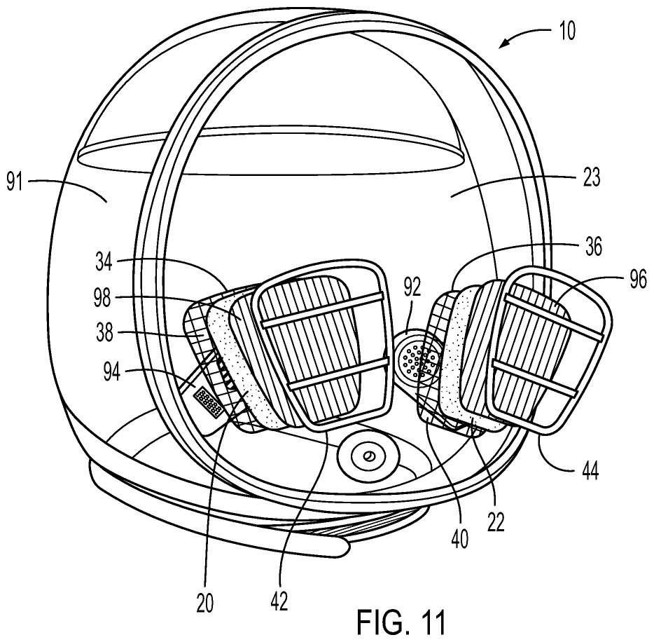

Referring now to FIGS. 10 and 11, additional secondary filters 96, 98 and one or more air pumps 92, 94 that may be incorporated with the encapsulation unit 10 is shown. The secondary filters 96, 98 provide an additional layer of filtration for the outside air entering the interior volume 91 of the head encapsulation unit 10. The one or more air pumps/fans 92, 94 may be incorporated to improve the ventilation within the interior volume 91 of the head encapsulation unit 10 by aiding the movement of air into and out of the unit. Since the air pumped by the one or more air pumps 92, 94 travel through one or more filters before reaching the interior volume 91 or the outside air, clean air enters and exits the head encapsulation unit 10. As such, the air breathed by the user of the head encapsulation unit 10 may be cleaned and transferred in and out of the head encapsulation unit 10 more fluidly. The one or more air pumps 92, 94 may also mitigate and prevent fogging from occurring on the inside or outside of the transparent layer 23. The one or more air pumps/fans 92, 94 are also designed to be attached to the head encapsulation unit 10 without puncturing or affecting the functioning of the filters, as described elsewhere herein.

The secondary filters 96, 98 may be different than the filters 20, 22 and may be incorporated in line with the cutouts 34, 36. Each cutout 34, 36 may have a secondary filter 96, 98 removably attached with the cutouts 34, 36. The filters 20, 22 may be first filters, and the secondary filters 96, 98 may be second filters. By way of example and not limitation, the first filters 20, 22 may be airborne particle trapping filters, specifically trapping infectious airborne particles such as viruses and bacteria, and the second filters 96, 98 may be a disinfectant filter. Alternatively, the second filters 96, 98 may be oxidizing filters or odor blocking filters in addition or in substitution of being a disinfectant filter. By way of example and not limitation, the first filters 20, 22 may be N95 or N99 filters and the second filter 96, 98 may be lined or covered with potassium permanganate.

The first filters 20, 22 and the second filters 96, 98 may be layered on top of each other and be between the mounting bracket 38 and mounting cover 42, and the mounting bracket 40 and the mounting cover 44. By way of example and not limitation, the first filters 20, 22 may be considered inner filters closest to the interior volume 91 and may be attached to the mounting brackets 38, 40. By way of example and not limitation, the second filters 96, 98 may be considered the outer filters that are closest to the outside environment and may be attached to the mounting covers 42, 44. Alternatively, the first filters 20, 22 and the second filters 96, 98 may switch places where the first filters 20, 22 may be the outer filters and the second filters 96, 98 may be the inner filters.

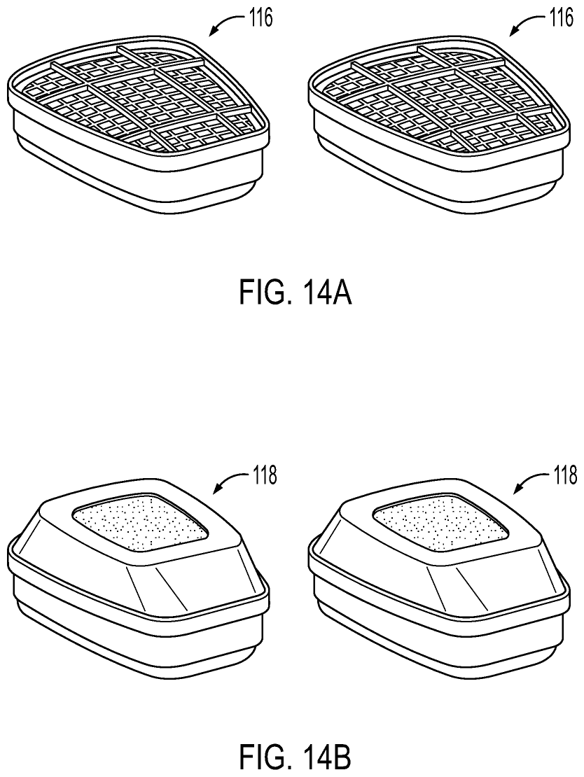

By way of example and not limitation, the second filters 96, 98 and/or the first filters 20, 22 may be replaced with a type of respirator cartridges 116, 118 shown in FIGS. 14A-B. The mounting covers 42, 44 may be removed in addition with the second filters 96, 98 and/or the first filters 20, 22 and instead either the first respirator cartridges 116 or the second respirator cartridges may be attached at the cutouts 34, 36. By way of example and limitation, the first respirator cartridges 116 may provide protection for the interior volume 91 (see FIG. 11) and the user from one or more organic vapor, acid gas, ammonia, methylamine, formaldehyde, and other volatile organic compounds. By way of example and not limitation, the second respirator cartridges 118 may provide protection for the interior volume 91 (see FIG. 11) and the user from one or more of lead and asbestos particles, welding fumes and mists, fiberglass dust, cadmium, arsenic, and MDA, and other dusts fibers, fumes, or mists. By way of example and not limitation, the second respirator cartridges 118 may be 3M PARTICULATE FILTER P100 or P2091 filter cartridge. By way of example and not limitation, one or more air pumps/fans 92, 94 (see FIG. 11) may be incorporated in combination of the respirator cartridges 116, 118 to transfer air between the interior volume 91 and the outside environment and through the filter cartridges. In a preferred example, the one or more air pumps 92, 94 may be attached to the interior of the head encapsulation unit 10 (see FIG. 11), as described elsewhere herein. As such, the one or more air pumps 92, 94 would less likely interfere with the air inlets of respirator cartridges located outside of the head encapsulation unit 10.

As shown in FIG. 10, one or more air pumps/fans 92, 94 may be attached to the head encapsulation unit 10 at the cutouts 34, 36 that regulate airflow to and from the interior volume 91 of the head encapsulation unit 10. The one or more air pumps 92, 94 may be attached to various components of the head encapsulation unit 10, such as the filters, mounting brackets 38, 40, or the mounting covers 42, 44. The one or more air pumps 92, 94 are designed to be attached to such structural components of the head encapsulation unit 10 without puncturing or affecting the functioning of the filters, as described elsewhere herein. The one or more air pumps 92, 94 may be an air blower pump/fan that pushes air, an air vacuum pump/fan that pulls and suctions air, or a combination thereof. There may only be one air pump 92, 94 attached at only one cutout 34, 36, or each cutout 34, 36 may have an air pump 92, 94 attached thereto. The one or more air pumps 92, 94 may be attached on the outside or inside of the interior volume 91, or a combination thereof. By way of example and not limitation, each of the air pumps 92, 94 may have a power setting, such as providing low, medium, or high airflow.

By way of example and not limitation, and as shown in FIG. 10, the one or more air pumps 92, 94 may be attached outside of the head encapsulation unit 10 and provide air flow force between the outside environment and the interior volume 10. By way of example and not limitation, each air pump 92, 94 may be attached on the outside surface of the filters 96, 98 or be attached to the mounting covers 42, 44. Since the air pumps 92, 94 are on the outside of the head encapsulation unit 10, and reachable by the user's hands, the user may manually activate and deactivate the pumps 92, 94 while wearing the unit. Being mounted to the outside of the head encapsulation unit 10 may also provide better intake airflow to the air pumps that are air blower pumps.

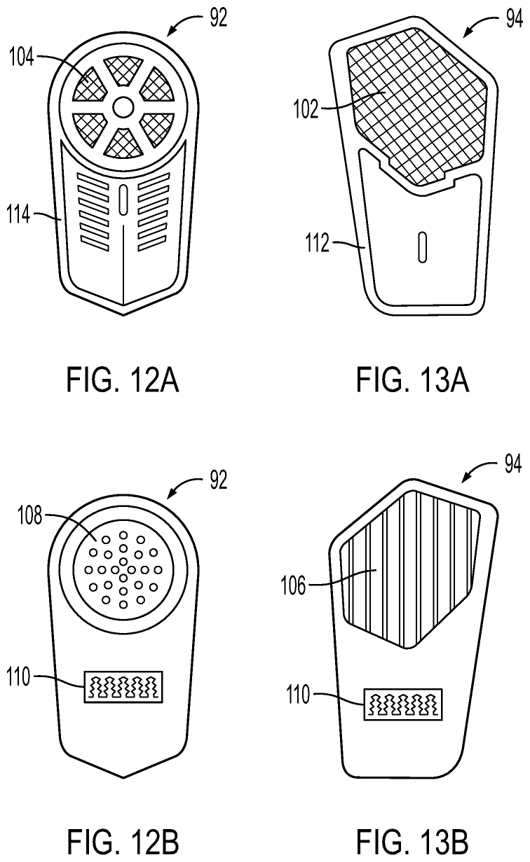

By way of example and not limitation, two air pumps 92, 94 may be attached outside the transparent layer 23 at the cutouts 34, 36. An air blower pump/fan 92 that pushes air inside the interior volume 91 may be attached outside of the transparent layer 23 and located at the second cutout 36. By way of example and not limitation, the air blower pump 92 may be attached such that the airflow output 108 (see FIG. 12B) of the pump is facing and contacting the outer filter, which may be either the first filter 22 or the second filter 96, and having an air intake 104 (see FIG. 12A) facing the outside environment to intake ambient air. Alternatively, the airflow intake 104 of the blower pump shown in FIG. 12A may be located on a side 114 of the air blower pump 92 instead of being on an opposite side to the airflow output 108. The air blower pump 92 may push air inside and increase the pressure of the interior volume 91 of the head encapsulation unit 10. Since the head encapsulation unit 10 is in the deployed position and may be substantially rigid, as described elsewhere herein, the change in pressure of the interior volume 91 may not change the shape of the body of the head encapsulation unit 10. As the air is blown by the air blower pump 92, airflow may first travel through the layers of filters that purifies the airflow, as described elsewhere herein. By way of example and not limitation, the air blower pump 92 may be directly attached to the outer filter or be attached to the mounting cover 44. By way of example and not limitation, the air blower pump 92 may be attachable to the outer filter or the mounting cover 44 using a clip or an adhesive compound. If the mounting cover 44 is metallic, the air blower pump 92 may be attachable to the mounting cover 44 using magnets. Alternatively, there may exist an interlocking mechanism between the mounting cover 44 and the air blower pump 92 for attachment. The interlocking mechanism may be a Velcro strap 110 (see FIG. 12B) on the air blower pump 92 and a corresponding Velcro strap on the mounting cover 44. The Velcro strap 110 shown in FIG. 12B may be replaced by a snap on button, clip, adhesive compound, or a magnetic strip, described elsewhere herein. The corresponding Velcro strap may be replaced by a snap on button, clip, adhesive compound, or a magnetic strip. The corresponding Velcro strap may instead of being located on the mounting cover 44 be located on the outer filter.

By way of example and not limitation, the second pump may be an air vacuum pump/fan 94 that may be attached outside of the transparent layer 23 and be located at the first cutout 34 to suction air from the interior volume 91. By way of example and not limitation, the air vacuum pump 94 may be attached such that the airflow intake 106 (see FIG. 13B) of the pump that pulls air from the interior volume 91 to the outside is facing and contacting the outer filter, which may be either the first filter 20 or the second filter 98. The air vacuum pump 94 may have an airflow output 102 (see FIG. 13A) facing the outside environment to release the pulled air from the interior volume 91 out to the outside environment. Alternatively, the airflow output 102 of the vacuum pump shown in FIG. 13A may be located on a side 112 of the air vacuum pump 94 instead of being on an opposite side to the airflow intake 106. The air vacuum pump 94 may suction air and reduce the pressure of the interior volume 91 of the head encapsulation unit 10. Since the head encapsulation unit 10 is in the deployed position and may be substantially rigid, as described elsewhere herein, the change in pressure of the interior volume 91 may not change the shape of the body of the head encapsulation unit 10. The reduction in pressure of the interior volume 91 caused by the air vacuum pump 94 may be balanced by the increase in pressure of the interior volume 91 caused by the air blower pump 92. The air vacuum pump 94 may pull the air of the interior volume 91 through the layers of filters that purifies the air, as described elsewhere herein, before releasing the air to the outside environment. The air vacuum pump 94 may be directly attached to the outer filter or be attached to the mounting cover 42, as described elsewhere herein.

The combination of the air blower pump 92 and the air vacuum pump 94 attached outside the transparent layer 23 and at the cutouts 34, 36 may improve the ventilation of airflow to and from the interior volume 91 of the head encapsulation unit 10. By way of example and not limitation, the mounting brackets 38, 40 that are the closest structural component near the interior volume 91 may have openings carved in their bodies to help the air pumps 92, 94 create better airflow to and from the interior volume 91. The openings may be a hole with the same dimension as the air output of the air blower pump 92 and of the air intake of the vacuum pump 94.

By way of example and not limitation, the air vacuum pump 94 may be omitted and only the air blower pump 92 may be attached outside of the transparent layer 23 and be located at the second cutout 36, as described elsewhere herein. The air blower pump 92 may push air through the filters to the inside of the interior volume 91 and create a positive pressure condition that makes the interior pressure greater than the pressure outside of the head encapsulation unit 91. As a result of the positive pressure condition, the first cutout 34 opening, which does not have a pump mounted, may serve as an exhaust opening that depressurizes the interior volume 91.

By way of example and not limitation, the air blower pump 92 may be omitted and only the air vacuum pump 94 may be attached outside of the transparent layer 23 and be located at the first cutout 34, as described elsewhere herein. The air vacuum pump 94 may pull air through the filters from the inside of the interior volume 91 to the outside environment and create a negative pressure condition that makes the interior pressure less than the pressure outside the head encapsulation unit 10. As a result of the negative pressure condition, the second cutout 36 opening, which does not have a pump mounted, may serve as an intake opening that balances the interior pressure with the exterior pressure.

In another example, and as shown in FIG. 11, the one or more pumps 92, 94 may all be attached inside the interior volume 91 and to the mounting brackets 38, 40. Since there exists spacing between the interior surface of the transparent layer 23 of the head encapsulation unit 10 and the face of the user wearing the unit, the pumps 92, 94 may be attached inside the interior volume 91. With the pumps 92, 94 attached inside the head encapsulation unit 10, there would be a less likely chance for the pumps 92, 94 to detach by outside forces and objects. By way of example and not limitation, the pumps 92, 94 may be remote controlled to operate the pumps inside the head encapsulation unit 10. By way of example and not limitation, the two air pumps 92, 94 may be an air blower pump 92 and an air vacuum pump 92. The vacuum air pump 92 may be attached at first cutout 34, and the air blower pump 92 may be attached at second cutout 36.

An air vacuum pump/fan 94 that pulls air from the outside environment may be attached inside of the transparent layer 23 and be located at the first cutout 34. The air vacuum pump 94 may be attached such that its airflow intake 106 (see FIG. 13B) that suctions air is facing and contacting the inner filter, which may be either the first filter 20 or the second filter 98, and the mounting bracket 38. The air vacuum pump 94 may have an airflow output 102 (see FIG. 13A) facing the interior volume 91 to pull air to the inside from the outside environment. Alternatively, the airflow output 102 of the vacuum pump shown in FIG. 13A may be located on a side 112 of the air vacuum pump 94 instead of being on an opposite side to the airflow intake 106. By way of example and not limitation, the mounting bracket 38 may have an opening carved in its body to allow the air vacuum pump 94 to contact the inner filter and create better suction flow from the outside environment to the interior volume 91. The openings may be a hole with the same dimension as the air intake of the air vacuum pump 94. The air vacuum pump 94 may suction air and increase the pressure of the interior volume 91 of the head encapsulation unit 10. The air vacuum pump 94 may pull the outside air through the layers of filters that purifies the air, as described elsewhere herein, before the air is suctioned into the interior volume 91. By way of example and not limitation, the air vacuum pump 94 may be directly attached to the mounting bracket 38 or the inner filter. By way of example and not limitation, the attachment to the mounting bracket 38 or the inner filter may be done using a clip or adhesive compound. If mounting bracket 38 is metallic, the air vacuum pump 94 may be attachable using magnets. Alternatively, there may exist an interlocking mechanism between the mounting bracket 38 and the air vacuum pump 94 for the attachment of the two components. The interlocking mechanism may be a Velcro strap 110 (see FIG. 13B) on the air blower pump 92 and a corresponding Velcro strap on the mounting bracket 38. The Velcro strap 110 shown in FIG. 13B may be replaced by a snap on button, clip, adhesive compound, or a magnetic strip, described elsewhere herein. The corresponding Velcro strap may be replaced by a snap on button, clip, adhesive compound, or a magnetic strip. The corresponding Velcro strap may instead of being located on the mounting bracket 38 be located on the inner filter.

An air blower pump/fan 92 that pushes air out of the interior volume 91 and to the outside environment may be attached to the inside of the transparent layer 23 and be located at the second cutout 36. The air blower pump 92 may be attached such that its airflow output 108 (see FIG. 12B) is facing and contacting the inner filter, which may be either first filter 22 or second filter 96, and the mounting bracket 40. The air blower pump 92 may have an air intake 104 (see FIG. 12A) facing the interior to intake air from the interior volume 91. Alternatively, the airflow intake 104 of the blower pump shown in FIG. 12A may be located on a side 114 of the air blower pump 92 instead of being on an opposite side to the airflow output 108. By way of example and not limitation, the mounting bracket 40 may have a carved opening, as explained elsewhere herein, for the output air pump and the inner filter to contact each other. The air blower pump 92 may push air from inside of the interior volume 91 to the outside environment and decrease the pressure of the interior volume 91. The reduction in pressure of the interior caused by the air blower pump 92 may be balanced by the increase in pressure of the interior caused by the air vacuum pump 94. The interior air pushed by the air blower pump 92 may first travel through the layers of filters that purifies the air, as described elsewhere herein, and then release to outside environment. By way of example and not limitation, the air blower pump 92 may be directly attached to the mounting bracket 40 or the inner filter, as described elsewhere herein.

By way of example and not limitation, the air blower pump 92 may be omitted and only the air vacuum pump 94 may be attached inside of the transparent layer 23 and located at the first cutout 34, as described elsewhere herein The air vacuum pump 94 may pull air through the filters from the outside environment towards the interior volume 91 to create a positive pressure condition that makes the interior pressure greater than the pressure outside the encapsulation unit 91. As a result of the positive pressure condition, the second cutout 36 opening, which does not have a pump mounted, may serve as an exhaust opening that depressurizes the interior.

By way of example and not limitation, the air vacuum pump 94 may be omitted and only the air blower pump 92 may be attached inside of the transparent layer 23 and be located at the second cutout 36, as described elsewhere herein. The air blower pump 92 may push air through the filters from the inside of the interior volume 91 to the outer environment and create a negative pressure condition that makes the interior pressure lower than the pressure outside the encapsulation unit. As a result of the negative pressure condition, the first cutout 34 opening, which does not have a pump mounted, may serve as an intake opening that balances the interior pressure with the exterior pressure.

In another example, one of the pumps/fans may be attached inside the interior volume 91, as described elsewhere herein, and the other pump/fan may be attached outside of the interior volume 91 of the head encapsulation unit 10, as described elsewhere herein. By way of example and not limitation, two air blower pumps 92 may be used, where one blower pump is attached inside the interior volume 91 and pushing air out to the outside environment and another blower pump is attached outside of the interior volume 91 and pushing air inside. By way of example and not limitation, two air vacuum pumps 94 may be used, where one vacuum pump is attached inside the interior volume 91 and suctioning air from the outside environment to the inside, and another vacuum pump is attached outside of the interior volume 91 and suctioning air from the outside environment to the inside of the head encapsulation unit 10. By way of example and not limitation, an air blower pump 92 may be attached outside of the interior volume 91 and pushing air inside, and an air vacuum pump 94 be attached inside and pulling air outwards to the outside environment. By way of example and not limitation, an air blower pump 92 may be attached inside the interior volume 91 and pushing air out, and an air vacuum pump 94 may be attached outside and pulling air inside the head encapsulation unit 10.

Although not explicitly shown in FIG. 10, the head encapsulation unit 10 may have other structural features describe elsewhere herein, for example the release valve 70 with a button 74 having frangible material 72, thin flexible material in front of the user's mouth 71, a microphone 80, a speaker 82, and other features.

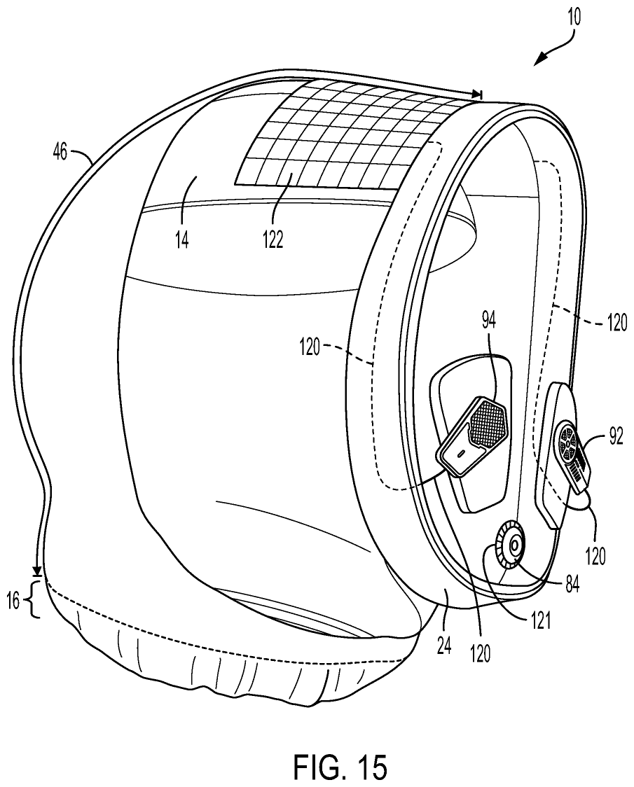

Referring now to FIG. 15, the incorporation of one or more solar panels with the head encapsulation unit is shown. The one or more solar panel strips 122 incorporated with the head encapsulation unit 10 may capture and convert solar energy to electrical energy to charge the batteries and power the one or more air pumps 92, 94. By way of example and not limitation, one or more solar panel strips 122 may be attached or integrated with the outside of the housing 46 of the head encapsulation unit 10. By way of example and not limitation, the one or more solar panel strips 122 may be thin-film solar panels in the form of amorphous silicon, cadmium telluride, or copper indium gallium thin-film solar panels. There may be between 1 to 12 thin-film solar panel strips 122 attached or integrated with the housing 46 of the head encapsulation unit 10. By way of example and not limitation, the one or more solar panel strips 122 may be miniature solar panels in the form of monocrystalline or polycrystalline miniature solar panels. The one or more solar panel strips 122 may be between 1 to 42 miniature solar panel modules attached or integrated with the housing 46 of the head encapsulation unit 10. By way of example and not limitation, the one or more solar panel strips 122 may be located on the portion of the outer surface of housing 46 located on top of the cap 14. By way of example and not limitation, the one or more solar panel strips 122 may be located on the left and right sides of the housing 46. By way of example and not limitation, the one or more solar panel strips 122 may be located on a rear portion of the housing 46 covering the back of the user's head. By way of example and not limitation, the solar panel strips 122 may be attached or integrated with the transparent portion of the housing 46. By way of example and not limitation, the solar panel strips 122 may be attached or integrated with the non-transparent portion of the housing 46.

Wirings 120 may be used to connect the solar panel strips 122 with the one or more air pumps 92, 94 to power or recharge the pumps. By way of example and not limitation, the wirings 120 may reach the one or more air pumps 92, 94 by traveling through wire conduits within the frame 24 of the head encapsulation unit 10. If the solar panel strips 122 are located on top of the cap 14, the wirings 120 may reach the cutouts near the lower region of the head encapsulation unit 10 and where the one or more air pumps 92, 94 are located by traveling through wire conduits within the frame 24. As such, the wirings 120 may provide the electrical energy generated by the solar panel strips 122 to the one or more air pumps 92, 94 to power or recharge them. Alternatively, the wirings 120 may not travel through the frame 24 and just be attached to an outer surface or inner surface of the head encapsulation unit 10. Although FIG. 15 shows the wirings 120 connecting the solar panel strips 122 to one or more air pumps 92, 94 outside of the head encapsulation unit 10, the wirings 120 may also reach the interior volume 91 if the air pumps are located therein, either directly or through the wire conduits within the frame 24. By way of example and not limitation, the wirings 120 may have a USB connector end designed to be connected to a USB port on the one or more air pumps 92, 94. By way of example and not limitation, the USB connection between the wirings 120 and the one or more air pumps 92, 94 may be USB-A, USB-B, USB-B Mini, USB-B Micro, or USB-C.

By way of example and not limitation, the one or more air pumps 92, 94 may be controlled using a smartphone via an application. The application on the smartphone may allow the user to turn on and off, adjust the power settings, and display the battery level of the air pumps 92, 94 attached to the head encapsulation unit 10. By way of example and not limitation, the application may allow the smartphone to adjust the pumping intensity of the air pumps 92, 94 between low, medium, or high intensity. By way of example and not limitation, the air pumps 92, 94 may communicate with the smartphone and the application via WI-FI or Bluetooth.

By way of example and not limitation, the one or more solar panel strips 122 may be in communication with the smartphone via the application. By way of example and not limitation, the one or more solar panel strips 122 may provide the smartphone, using the application, the amount of power and current that the panels are generating in real-time, and such information may be displayed on the smartphone. The application may use such information in conjunction with the battery levels of the air pumps 92, 94 to determine and display the duration of time to fully charge the air pumps 92, 94. By way of example and not limitation, the one or more solar panel strips 122 may communicate with the smartphone and the application via WI-FI or Bluetooth.

By way of example and not limitation, the filters and cartridges described elsewhere herein may communicate with the smartphone and the application via WI-FI and Bluetooth and indicate whether the air passage and percent purification has been compromised below a certain threshold level. The smartphone may monitor the filtering efficiency of the filters/cartridges using the application. Consequently, the user may then replace the filters/cartridges that are defective and no longer filter air correctly.

Alternatively, and by way of example and not limitation, the application may provide to the user when the filters and cartridges of the head encapsulation unit 10, described elsewhere herein, may need to be changed or replaced. By way of example and not limitation, the application may provide the filter/cartridge replacement notification on the smartphone after a certain period of time has passed, for example 1 to 12 months, from the usage of the originally installed filters/cartridges or from the time the filters/cartridges where previously replaced. The application may determine the time of usage of the originally installed filters/cartridges from when the application was first synced with the head encapsulation unit 10 or one of the components of the unit, such as the air pumps. The application may determine the time of usage of previously replaced filters/cartridges from the user providing such information directly to the application. The user may need to provide such information about the replacement of the filters in order to make a notification alert on the application disappear for the replacement of filters/cartridges and reset the filters/cartridges replacement time.

By way of example and not limitation, the head encapsulation unit 10 may have its own computing system that is connected to send and receive information to the air pumps 92, 94, solar panel strips 122, and filters/cartridges described elsewhere herein. By way of example and not limitation, the computing system of the head encapsulation unit 10 may communicate with the application downloaded on the smartphone via WI-FI or Bluetooth and provide the information pertaining to the air pumps 92, 94, solar panel strips 122, and filters/cartridges, described elsewhere herein, to the smartphone via the application. The application may be updated to add new features or to improve upon the features described herein regarding the usage of the application with the air pumps 92, 94, solar strips 122, filters, and the head encapsulation unit 10 in general. Although the application is described as being downloaded on a smartphone, the application may be downloaded and operated using any other mobile device.

By way of example and not limitation, one or more sensors 121 may be incorporated with the head encapsulation unit 10. By way of example and not limitation, the one or more sensors 121 may be located on the transparent layer 23 proximate to the fluid input port 84. Alternatively, the one or more sensors 121 may be located on any other part of the head encapsulation unit 10 described elsewhere herein. By way of example and not limitation, the one or more sensors 121 may be a biosensor, a light sensor, or a pressure sensor. By way of example and not limitation, the biosensor may detect airborne viruses, bacteria, vapor, dust, or toxic emission inside or outside of the head encapsulation unit 10. By way of example and not limitation, the light sensor may detect light in connection with operating the one or more solar panel strips 122. By way of example and not limitation, the pressure sensor may detect the pressure inside the head encapsulation unit. The information collected by the one or more sensors 121 may be sent and displayed on the smartphone via the software application for the user to determine the physical properties of the surrounding area and within the head encapsulation unit. The transmission of the information from the one or more sensors 121 to the smartphone may be conducted similar to what has been described elsewhere herein.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A head encapsulation unit wearable by a user for mitigating contact to airborne viruses, bacteria, vapor, dust, and toxic emissions, comprising:
a body defining an interior volume, the body including:
a bottom portion;
a transparent front panel having a first cutout and a second cutout; and
a top portion;
a first filter removably attachable to the first cutout and a second filter removably attachable to the second cutout; and
an air pump attached to the first filter to provide ventilation to the interior volume of the body.

2. The head encapsulation unit of claim 1, wherein the air pump is powered by a solar panel on the head encapsulation unit.

3. The head encapsulation unit of claim 1, wherein the air pump is a first air pump and a second air pump is attached to the second filter.

4. The head encapsulation unit of claim 3, wherein the first air pump is an air blower pump and the second air pump is an air vacuum pump.

5. The head encapsulation unit of claim 4, wherein the first air pump and the second air pump are attached outside of the interior volume of the body.

6. The head encapsulation unit of claim 4, wherein the first air pump and the second air pump are attached inside of the interior volume of the body.

7. The head encapsulation unit of claim 6, wherein the first air pump and the second air pump are configured to be remote controlled.

8. The head encapsulation unit of claim 1, further comprising a third filter removably attachable to the first cutout and a fourth filter removably attachable to the second cutout.

9. The head encapsulation unit of claim 8, wherein the first and second filters are designed to filter infectious airborne particles from entering the interior volume of the body and the third and fourth filters are disinfectant filters.

10. The head encapsulation unit of claim 9, wherein the first and second filters are closest to the interior volume of the body relative to the third and fourth filters.

11. The head encapsulation unit of claim 9, wherein the first and second filters are farthest away to the interior volume of the body relative to the third and fourth filters.

12. A head encapsulation unit wearable by a user for mitigating contact to airborne viruses, bacteria, vapor, dust, and toxic emissions, comprising:
a body defining an interior volume, the body including:
a bottom portion;
a transparent front panel having a first cutout and a second cutout; and
a top portion;
a first filter removably attachable to the first cutout and a second filter removably attachable to the second cutout;
a third filter removably attachable to the first cutout; and
a fourth filter removably attachable to the second cutout;
wherein the first and second filters are designed to filter infectious airborne particles from entering the interior volume of the body and the third and fourth filters are disinfectant filters.

13. The head encapsulation unit of claim 12, wherein the air pump is a first air pump and the mounting bracket is a first mounting bracket and a second air pump is attached to a second mounting bracket that is over the second filter.

14. The head encapsulation unit of claim 13, wherein the first air pump is an air blower pump and the second air pump is an air vacuum pump.

15. The head encapsulation unit of claim 14, wherein the first air pump and the second air pump are attached outside of the interior volume of the body.

16. The head encapsulation unit of claim 14, wherein the first air pump and the second air pump are attached inside of the interior volume of the body.

17. The head encapsulation unit of claim 16, wherein the first air pump and the second air pump are configured to be remote controlled.

18. The head encapsulation unit of claim 12, wherein the first and second filters are designed to filter infectious airborne particles from entering the interior volume of the body and the third and fourth filters are disinfectant filters.

19. The head encapsulation unit of claim 18, wherein the first and second filters are farthest away to the interior volume of the body relative to the third and fourth filters.

20. The head encapsulation unit of claim 12 wherein the third and fourth filters have potassium permanganate.
